(19) United States
(12) Patent Application Publication
(10) Pub. No.: US 2014/0243600 A1
(43) Pub. Date: Aug. 28, 2014

(54) OXYGEN MASK ADAPTABLE FOR UPPER GASTROINTESTINAL (UGI) ENDOSCOPY PROCEDURES PROVIDING ENHANCED OXYGEN CONCENTRATION TO MAINTAIN PATIENT OXYGENATION

(71) Applicant: Michelle Eisenberger, Colorado Springs, CO (US)

(72) Inventor: Michelle Eisenberger, Colorado Springs, CO (US)

(73) Assignee: Michelle Eisenberger, Colorado Springs, CO (US)

(21) Appl. No.: 14/188,471

(22) Filed: Feb. 24, 2014

Related U.S. Application Data
(60) Provisional application No. 61/769,677, filed on Feb. 26, 2013.

(51) Int. Cl. A61B 1/00; A61M 16/20; A61M 16/10; A61B 1/273; A61M 16/06

(52) U.S. Cl. CPC A61B 1/00147; A61B 1/2736; A61M 16/0683; A61M 16/1005; A61M 16/208
USPC 600/114

(57) ABSTRACT

An oxygen mask adaptable for upper gastrointestinal (UGI) endoscopy procedures providing enhanced oxygen concentration to maintain patient oxygenation. An oxygen mask in accordance with the present invention provides an aperture of sufficient dimensions to enable substantially unimpeded placement and manipulation of an endoscope during an endoscopic procedure while an overlying closure is in an open position and yet may also be closed to cover the aperture when the procedure is not being performed.

### CROSS REFERENCE TO PROVISIONAL PATENT APPLICATION

The present invention is related to, and claims priority from, U.S. Provisional Patent Application Ser. No. 61/769,677 filed Feb. 26, 2013 for “Endoscopic Oxygen Mask”, the disclosure of which is herein specifically incorporated in its entirety by this reference.

### BACKGROUND OF THE INVENTION

The present invention relates, in general, to the field of endoscopic procedures. More particularly, the present invention relates to an oxygen mask adaptable for upper gastrointestinal (UGI) endoscopy procedures providing enhanced oxygen concentration to maintain patient oxygenation.

An upper gastrointestinal (UGI) endoscopy, which may also be referred to as esophago-gastro-duodenoscopy (EGD), is a procedure enabling a physician to directly examine the upper part of the gastrointestinal (GI) tract. An endoscope, or gastroscope, comprising a flexible tube having a light source and lens enables the doctor to observe the upper GI tract on an associated monitor. The endoscope is generally utilized in conjunction with patient sedation or general anesthesia.

Exemplary of the current state of the art with respect to oxygen, or breathing, masks adapted for endoscopic procedures are U.S. Pat. No. 5,431,158 issued Jul. 11, 1995 to Tirotta for: “Endoscopy Breathing Mask”; U.S. Pat. No. 8,365,734 issued Feb. 5, 2013 to Lehman for: “Multi-Port, Intubation-Permitting, Oxygen Mask” and United States Patent Application Publication 2012/0330111 published Dec. 27, 2012 to Borody for “Mask for Use with a Patient Undergoing a Sedated Endoscopic Procedure”. Commercially available oxygen masks adapted for endoscopic procedures include The VBM Endoscopy Mask available from VBM Medizintechnik GmbH, Sulz, Germany and the Panoramic Oxygen Mask available from POM Medical, LLC, Simi Valley, Calif.

In each instance referenced above, the devices fail to provide a sufficiently large enough aperture in the mask to allow for substantially unimpeded manipulation of an endoscope while being worn by a patient and their use would result in undesirable rubbing of the scope with the periphery of the relatively small diameter aperture provided. Further, none of these devices provide for a trap door, or closure, which may be selectively opened (and optionally maintained in an open and/or closed position) during an endoscopic procedure and then re-closed upon completion of the procedure to assure a higher concentration of oxygen in order to maintain a patient's oxygen saturation during deep sedation or total intravenous general anesthesia while performing endoscopic airway procedures.

### SUMMARY OF THE INVENTION

Disclosed herein is an oxygen mask adaptable for upper gastrointestinal endoscopy procedures providing enhanced oxygen concentration to maintain patient oxygenation. Simply put, an oxygen mask in accordance with the present invention advantageously allows for administering oxygen to a patient while enabling substantially unimpeded performance of an UGI endoscopic procedure by the attending physician.

The oxygen mask disclosed herein provides for a higher concentration of oxygen to maintain a patient's oxygen saturation during deep sedation or total intravenous general anesthesia while performing an endoscopic airway procedure. Unlike conventional endoscopic oxygen masks, the aperture provided in accordance with the present invention will not interfere with the placement and manipulation of the endoscope.

Still further, the oxygen mask of the present invention can be inexpensively produced for single patient use, can be easily disposed of and does not require re-sterilization. The oxygen mask disclosed herein also allows for the escape of excess carbon dioxide due to the patient's respiration through facial vented openings in the mask structure while concurrently preventing exhaled carbon dioxide from accumulating in the mask reservoir bag.

Particularly disclosed herein is a breathing mask for providing oxygen to a patient during an endoscopic procedure which comprises an oxygen inlet and at least one respiration outlet; an aperture in the mask and a closure associated with the aperture selectively operable between a first aperture closed position and a second aperture open position, wherein the aperture is of sufficient dimensions to enable substantially unimpeded placement and manipulation of an endoscope in the patient when in the second aperture open position.

### BRIEF DESCRIPTION OF THE DRAWINGS

The aforementioned and other features and objects of the present invention and the manner of attaining them will become more apparent and the invention itself will be best understood by reference to the following description of a preferred embodiment taken in conjunction with the accompanying drawings, wherein:

FIG. 1 is a partial, front elevational view of a representative embodiment of an oxygen mask adaptable for upper gastrointestinal (UGI) endoscopy procedures in accordance with the principles of the present invention illustrating the trap door, or closure, in a closed position over the UGI endoscopy aperture;

FIG. 2 is a left side elevational view of the oxygen mask of FIG. 1 illustrating the closure (shown in phantom) in an aperture open position thereof; and

FIG. 3 is a right side elevational view of the oxygen mask of the preceding figures illustrating the closure being retained in an aperture open position to enable substantially unimpeded placement and manipulation of an endoscope in a patient.

### DESCRIPTION OF A REPRESENTATIVE EMBODIMENT

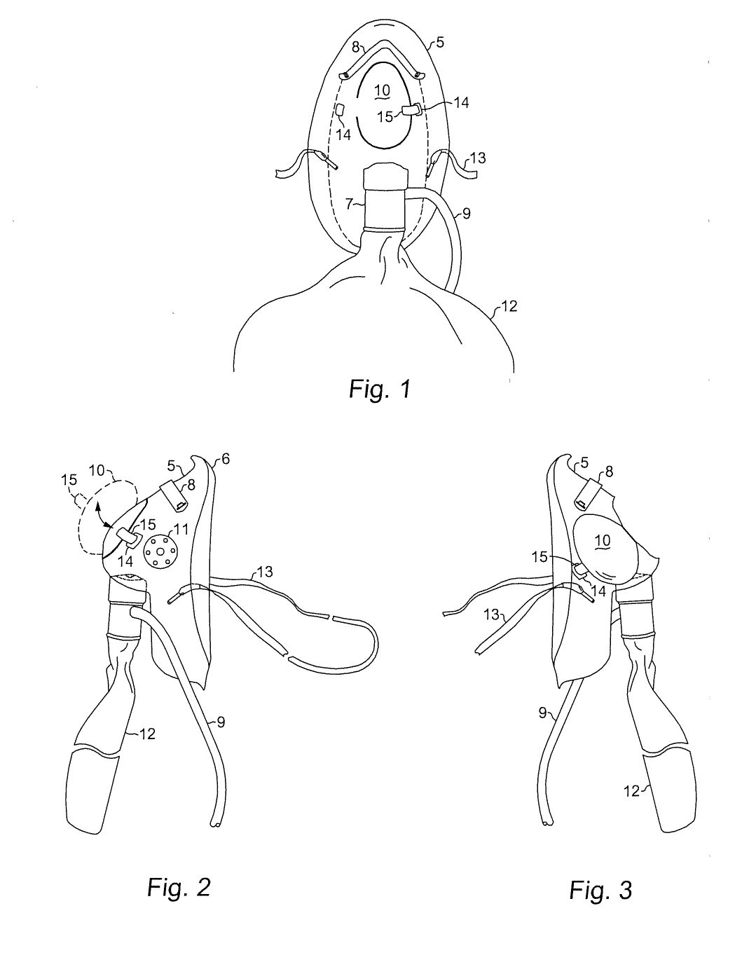

With reference now to FIG. 1, a front elevational view of a representative embodiment of an oxygen mask 5 adaptable for upper gastrointestinal (UGI) endoscopy procedures in accordance with the principles of the present invention is shown illustrative of the trap door, or closure 10, in a closed position over the endoscopy aperture. In a preferred embodiment, the closure 10 and corresponding aperture may be substantially 1.25 inches in width and approximately 1.75 inches in length.

The mask 5 is substantially oval in shape and preferably includes an out-turned flange 6 (FIG. 2) at its periphery with the edges being turned up away from contact with the patient's face thereby providing a rounded corner and a smooth contour to the face. The arched portion of the mask presents a shorter radius of curvature for adjoining the bridge of the patient's nose and may further comprise a flexible metal bridge 8 over the nasal area to enable form fitting to the features of a particular patient.

As shown, the mask 5 is coupled to an oxygen source (not shown) by means of connection tubing 9. A one-way valve 7 is also provided in addition to an oxygen reservoir bag 12. A strap 13, which comprises an elastic material with a generally flat cross-section serves to maintain the mask 5 in position over the patient's face and extends behind the face and below the ears.

The mask 5 comprises an elastomeric material such as a soft vinyl and the like which enables the closure 10 overlying the aperture to be hingedly connected to the mask structure at one edge by the flexible material itself to enable it to be moved between an aperture closed position as shown in FIG. 1 to an open position as shown in the succeeding figures. A hook and loop tab 15 and associated pads 14 may be provided to maintain the closure 10 in either position.

With reference additionally now to FIG. 2, a left side elevational view of the oxygen mask 5 of FIG. 1 is shown illustrating the closure 10 (shown in phantom) in an aperture open position thereof. By separating the tab 15 from the pad 14 adjacent the closure 10 open edge, the closure 10 may be selectively removed from over the mask 5 aperture to enable an endoscopic procedure to be performed.

With reference additionally now to FIG. 3, a right side elevational view of the oxygen mask 5 of the preceding figures is shown illustrating the closure 10 being retained in an aperture open position to enable substantially unimpeded placement and manipulation of an endoscope in a patient. The closure 10 may be selectively maintained in an aperture open position by affixing the tab 15 to the pad 14 adjacent to the hinged portion of the closure 10 during an endoscopic procedure. Upon completion of the endoscopic procedure, the tab 15 may be removed from that pad 14 and then secured to the opposing pad 14 as shown in FIG. 1.

As shown particularly with respect to FIG. 2, the mask 5 may advantageously also comprise ventilation holes 11 on one or both sides of the mask 5. These ventilation holes 11 serve to effectuate the exhalation of excess gases. In operation, the mask 5 in accordance with the present invention serves to maintain a patient's oxygen saturation level while an endoscopic procedure is being performed yet providing an aperture of sufficient dimensions to enable substantially unimpeded placement and manipulation of an endoscope during an endoscopic procedure.

While there have been described above the principles of the present invention in conjunction with specific apparatus, it is to be clearly understood that the foregoing description is made only by way of example and not as a limitation to the scope of the invention. Particularly, it is recognized that the teachings of the foregoing disclosure will suggest other modifications to those persons skilled in the relevant art. Such modifications may involve other features which are already known per se and which may be used instead of or in addition to features already described herein. Although claims have been formulated in this application to particular combinations of features, it should be understood that the scope of the disclosure herein also includes any novel feature or any novel combination of features disclosed either explicitly or implicitly or any generalization or modification thereof which would be apparent to persons skilled in the relevant art, whether or not such relates to the same invention as presently claimed in any claim and whether or not it mitigates any or all of the same technical problems as confronted by the present invention. The applicants hereby reserve the right to formulate new claims to such features and/or combinations of such features during the prosecution of the present application or of any further application derived therefrom.

As used herein, the terms “comprises”, “comprising”, or any other variation thereof, are intended to cover a non-exclusive inclusion, such that a process, method, article, or apparatus that comprises a recitation of certain elements does not necessarily include only those elements but may include other elements not expressly recited or inherent to such process, method, article or apparatus. None of the description in the present application should be read as implying that any particular element, step, or function is an essential element which must be included in the claim scope and THE SCOPE OF THE PATENTED SUBJECT MATTER IS DEFINED ONLY BY THE CLAIMS AS ALLOWED. Moreover, none of the appended claims are intended to invoke paragraph six of 35 U.S.C. Sect. 112 unless the exact phrase “means for” is employed and is followed by a participle.

1. A breathing mask for providing oxygen to a patient during an endoscopic procedure comprising:
an oxygen inlet in said mask;
at least one respiration outlet in said mask;
an aperture in said mask; and
a closure associated with said aperture selectively operable between a first aperture closed position and a second aperture open position, said aperture being of sufficient dimensions to enable substantially unimpeded placement and manipulation of an endoscope in said patient when in said second aperture open position.

2. The breathing mask of claim 1 further comprising:
an oxygen reservoir associated with said oxygen inlet.

3. The breathing mask of claim 1 further comprising a strap for securing said mask to the face of said patient.

4. The breathing mask of claim 3 wherein said strap comprises an adjustable elastic strap.

5. The breathing mask of claim 1 wherein said closure is hingedly attached to said mask adjacent said aperture.

6. The breathing mask of claim 1 further comprising:
structure for retaining said closure in said first aperture closed position.

7. The breathing mask of claim 6 wherein said structure comprises a hook and loop fastener.

8. The breathing mask of claim 1 further comprising:
structure for retaining said closure in said second aperture open position.

9. The breathing mask of claim 8 wherein said structure comprises a hook and loop fastener.

10. The breathing mask of claim 1 wherein said aperture is substantially 1.75 inches in length.

11. The breathing mask of claim 1 wherein said aperture is substantially 1.25 inches in width.

12. The breathing mask of claim 2 wherein said oxygen reservoir further comprises a one-way valve intermediate said oxygen reservoir and said oxygen inlet.

13. The breathing mask of claim 1 further comprising:
a flexible metal bridge on said mask adjoining a nasal area thereof.

14. The breathing mask of claim 1 wherein said breathing mask is formed of an elastomeric material.

15. The breathing mask of claim 14 wherein said elastomeric material comprises a soft vinyl.

16. The breathing mask of claim 1 wherein said breathing mask is disposable following use by said patient and said endoscopic procedure.
